(19) United States
(12) Patent Application Publication
(10) Pub. No.: US 2016/0158425 A1
(43) Pub. Date: Jun. 9, 2016

(54) APPARATUS FOR BLOOD CONCENTRATION

(71) Applicant: BRIGHTWAKE LIMITED, Nottingham Nottinghamshire (GB)

(72) Inventors: Stephen COTTON, Nottingham, Nottinghamshire (GB); Terence GOURLAY, Erskine, Renfrewshire (GB)

(21) Appl. No.: 14/904,905

(22) PCT Filed: Jul. 23, 2014
(86) PCT No.: PCT/GB2014/052249

§ 371 Date: Jan. 13, 2016

(30) Foreign Application Priority Data
Jul. 23, 2013 (GB) 1313137.0

(51) Int. Cl. A61M 1/02; A61M 1/36; B01D 71/50; A61M 1/10

(52) U.S. Cl. CPC A61M 1/0281; A61M 1/1039; A61M 1/3635; A61M 1/3679; B01D 71/50; A61M 2205/75; A61M 2202/0413; A61M 2205/02

(57) ABSTRACT

A disposable blood transfer conduit is used in association with a pump drive unit, a blood collection vessel and a blood concentration device. The blood transfer conduit comprises a blood transfer tube (13) and a peristaltic pump element (12) having an enclosed housing mounted about the blood transfer tube (12). At least part of the blood transfer tube (13) is resiliently deformable and extends through the enclosed housing of the peristaltic pump element (12). The disposable blood transfer conduit may be used in the concentration of blood for autologous blood transfusion.

The present invention relates to apparatus for use in concentrating blood. In particular, the present invention relates to apparatus for use in the concentration of blood for autologous blood transfusion.

Autologous blood transfusion is the process by which blood is collected from and retransfused back to the same patient. Blood may be collected either prior to or during surgery for retransfusion when required, often during or at the end of the surgical procedure. With donated blood generally being in short supply, there is increasing interest in autologous blood transfusion and methods for recovering blood which would traditionally have been lost to surgical drainage.

Autologous blood transfusion has a number of advantages over allogenic blood transfusion (the transfusion of blood from an unrelated donor), including there being no risk of the blood being incompatible, and no risk of transferring a blood-borne disease from person to person. It is most commonly used in surgery in which the likelihood of requiring a blood transfusion is high, such as surgery on the heart, blood vessels, bones and chest.

Where blood for use in autologous blood transfusion is collected from the patient prior to surgery, collection is carried out in the same manner as in conventional blood donation. However, where collection is carried out during surgery a suction system is typically used to collect the blood before it is filtered and concentrated, ready for storage or retransfusion.

Conventionally, a centrifuge system is used to concentrate the collected blood by separating the red blood cells from plasma and other blood components. This forms a red blood cell concentrate for transfusion. Passive systems for the concentration of blood are also known. For instance, WO20091141589 discloses apparatus for separating components of a fluid such as blood in which a superbsorbent material is used to draw blood plasma from whole blood, through a porous membrane. Cellular material, such as red blood cells, is too large to pass through the membrane and therefore forms a red blood cell concentrate that can be stored or retransfused back into the patient.

Complete systems for the collection and processing of blood are known, and enable collection, filtration and concentration of blood to take place in situ and with minimal handling. Such systems typically comprise a number of components, and may include a chamber in which the blood is initially collected, a device for concentrating the blood, and means for transferring the collected blood between the two. The transfer of blood between components is conventionally carried out either by a pump or under vacuum. This transfer of blood can present a number of difficulties, including the frequent need for sanitisation of the lines and equipment to prevent cross-contamination, and the risk of cells becoming damaged as the blood is collected and processed.

There has now been devised improved apparatus for use in concentrating blood, which overcomes or substantially mitigates the above mentioned and/or other problems associated with the prior art.

According to a first aspect of the invention there is provided a disposable blood transfer conduit for use in association with a pump drive unit, a blood collection vessel and a blood concentration device,
wherein the conduit comprises a blood transfer tube and a peristaltic pump element having an enclosed housing mounted about the blood transfer tube,
a first end of the blood transfer conduit being adapted for connection, in use, to the blood collection vessel and a second end of the blood transfer conduit being adapted for connection, in use, to the blood concentration device,
wherein at least part of the blood transfer tube is resiliently deformable and extends through the enclosed housing of the peristaltic pump element, and wherein the housing of the peristaltic pump element is adapted for releasable engagement, in use, with the pump drive unit to convey blood from the blood collection vessel to the blood concentration device.

By a “peristaltic pump element” is meant a unit that, when coupled to an external drive unit, constitutes a peristaltic pump. A peristaltic pump is a type of positive displacement pump used for pumping fluids. The fluid passes through a flexible tube, which is fitted within a pump casing. Compression of the tube by the pump and displacement of the point of compression along the tube causes the fluid to be propelled through the tube, in an action known as peristalsis.

In a peristaltic pump, the liquid being pumped is contained wholly within the flexible tube and does not come into contact with the pump itself.

In addition, peristaltic pump units have few moving parts and are driven by an external drive unit, meaning that they are relatively low cost items with little need for maintenance. The limited number of moving parts and lack of need for valves or seals in the pump also reduces the likelihood of a fault or mechanical problem arising.

The blood transfer conduit of the invention is disposable. By “disposable” is meant, in the context of the invention, that the blood transfer conduit is intended to be used only once and then disposed of and replaced. The use of a disposable blood transfer conduit reduces and/or removes the need for cleaning and sterilising of the conduit between uses, removing the risk of cross-contamination between patients.

The blood transfer conduit of the invention is advantageous in that it comprises a peristaltic pump element that is mounted about the blood transfer tube and can be easily coupled to the pump drive unit. The peristaltic pump element can be manufactured from inexpensive moulded plastics components, the more costly components of the peristaltic pump being incorporated into the pump drive unit and being reusable. The housing of the peristaltic pump element may be engaged with the pump drive unit simply by pressing the housing onto a projecting drive shaft of the pump drive unit, and may be removed from the pump drive unit by the reverse action. This facilitates use of the blood transfer conduit, as there is no need for the blood transfer tube to be fitted around the components of the pump that, in use, bear against the tube in order to exert a peristaltic action. The blood transfer conduit can therefore be engaged with, and disengaged from, the associated apparatus very easily and rapidly, and the inexpensive nature of the peristaltic pump element means that it can economically be disposed of together with the blood transfer tube.

The blood transfer conduit of the present invention conveniently comprises a tube of plastics material, one end of which is adapted for connection, in use, to a blood collection vessel and the other end of which is adapted for connection, in use, to a blood concentration device, and a peristaltic pump element having an enclosed housing mounted about the tube at a point intermediate the ends of the tube. At least that portion of the tube about which the housing of the peristaltic pump element is fitted is resiliently deformable.

Peristaltic pumps operate by mechanical compression of a tube through which fluid is to be pumped and displacement of the point of compression along the tube. Compression of the tube may entail entirely occluding the tube, or only partial occlusion of the tube. Preferably, in the present invention the peristaltic pump element is such that, in use, the tube is partially occluded but not entirely occluded. This is advantageous as total occlusion of the tube may damage cells carried within the blood. The degree of occlusion may be such that the cross-sectional area of the tube is reduced by between 50% and 95%, or between 75% and 95%.

The housing of the peristaltic pump element conveniently defines a circular cavity with two openings, such that the blood transfer tube extends through one of the two openings, around the internal surface of the cavity, and exits the housing through the other opening. Typically, the openings are disposed such that the tube extends around the internal wall of the cavity, through at least 180® of arc and more typically through about 270° of arc. The housing typically has the form of a drum or cylinder, with the openings formed in the cylindrical surface. Typically, the diameter of the drum or cylinder is greater than its depth.

Compression elements are arranged within the cavity such that, in use, they bear upon the resiliently deformable tube in order to exert a pumping action upon fluid contained within the tube.

Most conveniently, the compression elements are freely mounted rollers that are held in place within the cavity simply by virtue of being dimensioned such that they fit closely into the space between the blood transfer tube and the internal wall of the cavity between the openings.

Preferably, three such rollers are present and are held in close abutment, such that each roller contacts each of the other two with a central space between them.

The base of the housing, ie the face of the housing that abuts the pump drive unit when the peristaltic pump element is engaged with the pump drive unit, is preferably provided with an aperture through which, in use, a drive shaft is inserted. The aperture is typically circular and is aligned with the space between the rollers. In use, a drive shaft of the pump drive unit is inserted into the housing, through the aperture, and frictionally engages the rollers. The rotating drive shaft thereby acts upon the rollers, causing them to rotate within the cavity and to exert a peristaltic pumping action upon the blood transfer tube.

In use, a first end of the blood transfer conduit is connected to a blood collection vessel, and a second end of the blood transfer conduit is connected to a blood concentration device. Suitable connections for use in fluid-containing systems are known in the art, and any suitable means of connection may be used in the present invention. For example, the blood transfer conduit may be connected to the blood connection vessel and/or the blood concentration device via a screw fitting, a bayonet fitting, a clip, or a push-fit connector. A further seal, eg a rubber seal, may be present at the connection between the blood transfer conduit and the blood collection vessel and/or the blood transfer conduit and the blood concentration device, in order to ensure that the system is fluid tight.

The blood transfer tube comprises a tube, at least part of which is resiliently deformable. By “resiliently deformable” is meant, in the context of the invention, that the tube may be compressed or deformed under pressure and returns to its original dimensions when the pressure is removed. The blood transfer tube may be formed entirely from a resiliently deformable material, or may comprise a portion formed form a resiliently deformable material, the portion which is formed from a resiliently deformable material passing through the peristaltic pump element.

Where only part of the blood transfer tube is resiliently deformable, the resiliently deformable portion may be located between two non-resiliently deformable portions. The non-resiliently deformable portions of the blood transfer tube may be rigid or flexible, but are preferably flexible.

Suitable materials for use in the manufacture of tubes for conveying fluids are known in the art, and any suitable material may be used in the manufacture of the blood transfer tube. Preferably, the blood transfer tube is made of plastics material. All materials are preferably of medical grades.

The length of the blood transfer tube is sufficient that it can extend from the blood collection vessel to the blood concentration device. The diameter of the tube may be uniform or non-uniform, eg the diameter of the resiliently deformable portion may be the same as or different to the diameter of any non-resiliently deformable portions, but will typically be such that blood can flow easily through the tube. Preferably, the blood transfer tube has a diameter of from about 0.3 cm to 1.5 cm, or of from about 0.4 cm to 1.3 cm. More preferably, the blood transfer tube has a diameter of from about 0.5 cm to 1 cm.

The peristaltic pump element is adapted for releasable engagement, in use, with a pump drive unit. Conveniently, a drive shaft extends from a wall of the pump drive unit, and the pump drive unit is configured such that the peristaltic pump element may be fitted about the drive shaft, by inserting the drive shaft through the window in the base of the housing of the peristaltic pump unit. Thus, the peristaltic pump element may be engaged with the pump drive unit simply by pressing the housing of the peristaltic pump element onto the protruding drive shaft. The pump drive unit may have suitable formations to hold the peristaltic pump element in place and in its desired position and orientation.

In use, blood is generally collected from the patient and transferred into the blood collection vessel via a suction tube. Typically a source of reduced pressure, such as a vacuum pump, is used to generate suction. Before being deposited in the blood collection vessel, or once in the blood collection vessel, the blood may be mixed with an anticoagulant, such as heparin, in order to prevent clotting. The blood may be mixed with an anticoagulant in the suction tube, or the anticoagulant may be added to the blood collection vessel.

The blood collection vessel with which the blood transfer conduit of the present invention is used may be essentially conventional in form. Such a vessel typically has the form of a beaker, typically of clear, rigid plastic. Blood is generally introduced to such a vessel via a coarse filter to remove relatively large solid material such as bone fragments or the like. The vessel is preferably provided with an outlet port to which the blood transfer conduit of the invention may be connected. The blood collection vessel is of a suitable size to contain the blood collected, and typically has a volume of between about 0.5 and 3 L. Preferably, the blood collection vessel has a volume of between about 1 and 2.5 L, or of between about 1.5 and 2.5 L.

Devices for the concentration of blood are known in the art, and the blood concentration device used in conjunction with the blood transfer conduit of the invention may be any suitable blood concentration device. For example, the blood concentration device may be one which produces a red blood concentrate through centrifugation, or one which produces a red blood concentrate through passive means.

A suitable passive blood concentration device which may be used in conjunction with the blood transfer conduit of the invention is of the type disclosed in WO2009/141589. A particular blood concentration device of that general type is described in WO2011/061533 and comprises a blood bag which contains a porous membrane and a superabsorbent material. In use, blood is deposited in the blood bag, separated from the superabsorbent material by the membrane. Plasma and other unwanted blood products pass through the membrane and into the superabsorbent material, while the pore size of the membrane prevents the passage of cellular components. The bag may be gently agitated to prevent blood components occluding the pores in the membrane. Once sufficient plasma has been removed by the superabsorbent material, the remaining red blood cell concentrate can be transferred to a suitable container for storage or transfusion.

The porous membrane may form an inner bag disposed within the blood bag and containing the superabsorbent material. Thus, in accordance with WO2011/061533, the blood concentration device may comprise an outer bag formed of an impermeable material, an inner bag formed of a permeable material and containing a superabsorbent material, wherein the inner bag is fastened to, and suspended within, the outer bag.

The materials used to form the inner and outer bags are preferably flexible, to enable the bags to expand to accommodate fluid. The outer bag may be formed of any suitable material, but preferred materials are tough and impermeable to reduce the risk of fluid contained within the device from leaking out or becoming contaminated. The outer bag is preferably formed of sheets of synthetic plastic, such as polyethylene, polyamide, polypropylene, polyurethane, polyester or polycarbonate. A particularly preferred material for the outer bag is polyvinylchloride (PVC) in sheet form.

The outer bag is preferably provided with a port by which it can be connected to the blood transfer conduit of the invention.

The inner bag may be formed of any suitable material able to form a porous layer which allows liquid medium to pass through without the inner bag losing its integrity. The inner bag is preferably formed of sheets of synthetic plastic, such as polyethylene, polyimide, polypropylene, polyurethane, polyester or polyvinylchloride (PVC). One particularly preferred material for the inner bag is porous polycarbonate membrane.

The porous material of the inner bag allows liquid, eg blood plasma, to pass through, but substantially prevents the passage of cellular material. The inner bag is preferably completely sealed so the liquid is only able to enter the inner bag by passing through its porous walls.

The diameter of the pores in the inner bag typically ranges from 0.01 μm to 2 μm, more particularly 0.01 μm to 1 μm, and most particularly 0.1 pm to 1 μm, For use in the concentration of blood, the pores of the inner bag should have a diameter of no greater than about 1 μm in order to retain substantially all the particulate matter of the blood in the cavity between the outer bag and the inner bag.

Superabsorbent materials for use in the present invention are typically polymers that are capable of absorbing and retaining extremely large quantities of fluid relative to their own mass. Typically, such materials absorb aqueous solutions through hydrogen bonding with water molecules, and may absorb up to 200, 400, or 500 times or more their weight of water.

Amongst the most commonly used superabsorbent polymers are polyacrylates, ie salts of polyacrylic acid. For instance, the sodium salt of polyacrylic acid (cross-linked sodium polyacrylate) may be produced by the polymerization of acrylic acid blended with sodium hydroxide in the presence of an initiator.

Other superabsorbent polymers include polyacrylamide copolymer, ethylene maleic anhydride copolymer, cross-linked carboxymethylcellulose, polyvinylalcohol copolymers, cross-linked polyethylene oxide, starch-grafted copolymers of polyacrylonitrile, and others.

Another class of superabsorbent polymer that may be used in the invention is alginate, ie salts of alginic acid. Such material occurs naturally as a viscous gum that is abundant in the cell walls of brown algae, and commercial forms are extracted from seaweed. Alginic acid is a linear copolymer with homopolymeric blocks of (1-4)-linked β-D-mannuronate and its C-5 epimer α-L-guluronate residues, covalently linked together in different sequences or blocks. Alginates that are particularly suitable for use in the present invention are calcium alginate and sodium alginate.

The disposable blood transfer conduit of the invention may be supplied separately from the blood collection vessel and blood concentration device. Alternatively, the disposable blood transfer conduit may be supplied in combination with the blood collection vessel and/or the blood concentration device.

Thus, according to a second aspect of the invention there is provided a system for the concentration of blood for use in autologous blood transfusion, the system comprising a blood collection vessel and a blood concentration device, the blood collection vessel and blood concentration device being connected together by the disposable blood transfer conduit of the first aspect of the invention. The system may further comprise a pump drive unit for actuating the peristaltic pump element that forms part of the blood transfer conduit. Where the blood concentration device is a passive device such as a blood concentration bag as described above, the pump drive unit may be integrated with a means for agitating the blood concentration device.

According to a third aspect of the invention there is provided a method of concentrating blood for use in autologous blood transfusion, comprising collecting blood in a blood collection vessel, and transferring the blood from the blood collection vessel to a blood concentration device via a disposable blood transfer conduit of the first aspect of the invention.

Typically, blood will be collected in the blood collection vessel via a suction tube. The peristaltic pump element, connected to the pump drive unit, is then used to draw the blood through the blood transfer tube, from the blood collection vessel and into the blood concentration device. The blood concentration device is used to remove excess blood components, forming a red blood cell concentrate for storage or retransfusion.

According to a fourth aspect of the invention, there is provided a method of transferring blood between a blood collection vessel and a blood concentration device, comprising transferring the blood through a disposable blood transfer conduit according to the first aspect of the invention.

It will be appreciated that the blood transfer conduit, blood collection vessel and blood concentration device forming part of the second, third and fourth aspects of the invention may have any of the features described in relation to the first aspect of the invention.

An embodiment of the invention will now be described in greater detail, by way of example only, with reference to the accompanying drawings, in which

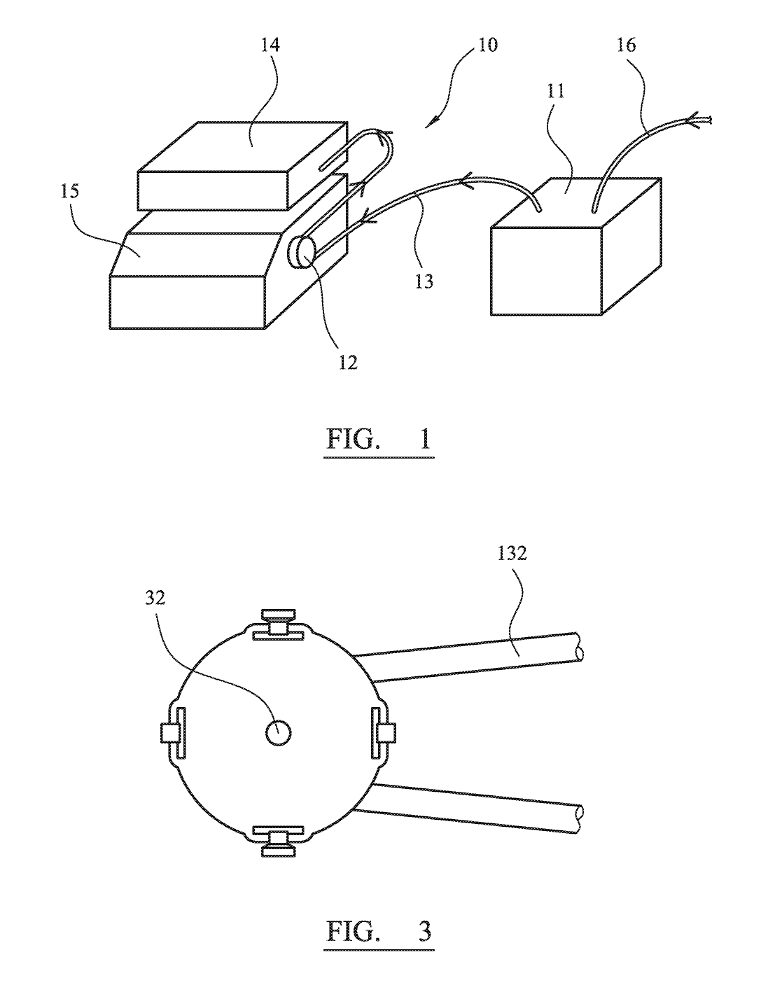

FIG. 1 shows a schematic drawing of a blood processing system comprising a disposable blood transfer conduit according to the invention;

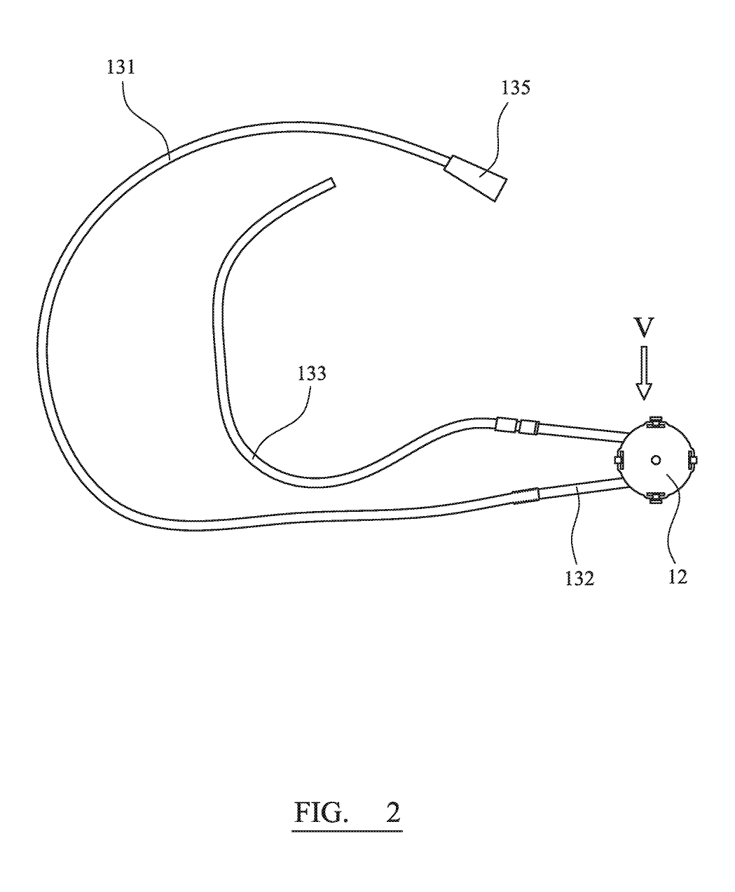

FIG. 2 shows a the disposable blood transfer conduit according to the invention that forms part of the system of FIG. 1;

FIG. 3 shows an underside plan view of a peristaltic pump element and resiliently deformable blood transfer tube, which form part of a disposable blood transfer conduit of FIG. 2;

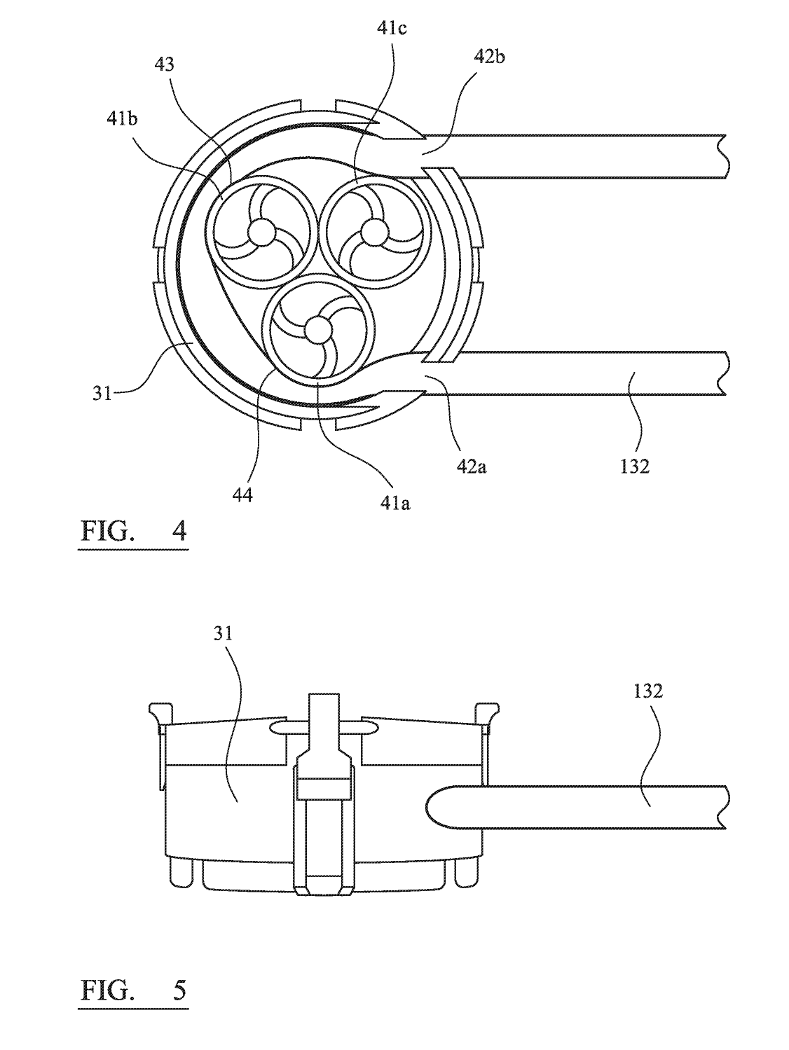

FIG. 4 shows an internal view of the peristaltic pump element of FIG. 3; and

FIG. 5 shows a side view of the peristaltic pump element and resiliently deformable blood transfer tube, as viewed along arrow V in FIG. 2.

Referring first to FIG. 1, a blood processing system 10 comprises a blood collection chamber 11, a blood transfer conduit according to the first aspect of the invention, which in turn comprises a blood transfer tube 13 about which is mounted a peristaltic pump element 12, and a blood concentration device in the form of a blood concentration bag 14 of the type described in WO2011/061533. The blood concentration bag 14 is mounted on a shaker unit 15. The blood collection chamber 11 is connected to the blood concentration bag 14, via the blood transfer conduit. The direction of blood flow is indicated by the arrows on the blood transfer tube 13.

The blood transfer conduit is shown in FIG. 2. As illustrated in that Figure, the blood transfer tube 13 comprises three portions: a first clear plastics tube 131, a resiliently deformable, elastomeric section 132 and a second clear plastics tube 133. The three portions of the tube 13 are connected by suitable joints to form a continuous conduit. The free end of the first clear plastics tube 131 carries a connector 135 by which the tube 13 may be connected to the blood collection vessel 11. Similarly, the free end of the second clear plastics tube 133 is configured for connection to the blood concentration bag 14. The peristaltic pump element 12 is mounted about the elastomeric section 132 of the tube 13.

The peristaltic pump element 12 is shown in greater detail in FIGS. 3, 4 and 5. The peristaltic pump element 12 comprises a cylindrical outer casing 31, the base of which is formed with a central, circular aperture 32 (see FIG. 3). As can be seen in the internal view of FIG. 4, an inlet 42a and an outlet 42b are provided in the curved wall of the cylindrical casing 31, approximately 90° apart. The elastomeric section 132 of the blood transfer tube passes through the inlet 42a, around the internal surface of the casing 31 and through the outlet 42b. Three freely mounted, cylindrical rollers 41a,41b,41c (see FIG. 3) fit closely into the space between the elastomeric tube section 132 and the internal wall of the casing 31 between the inlet 42a and the outlet 42b. The rollers 41a,41b,41c are pressed together by the resilience of the elastomeric tube section 132, such that each of the rollers 41a,41b,41c abuts each of the other two. Depending on the positions of the rollers 41a,41b,41c within the housing 31, at least two of them are in contact with the elastomeric tube section 132, and compress and partially occlude the elastomeric tube section 132 at their points of contact with it.

The central space between the rollers 41a,41b,41c is aligned with the aperture 32 in the base of the casing 31. In use, the peristaltic pump element 12 is fitted onto a drive shaft (not visible in the drawings) that protrudes from the side wall of the shaker unit 15. The drive shaft passes through the aperture 32 into the space between the rollers 41a,41b,41c and frictionally engages the rollers. Rotation of the drive shaft (clockwise as viewed in FIG. 4) causes the rollers 41a,41b,41 to rotate about their axes and to move clockwise within the housing 31. This motion causes the points of contact 43, 44 of the rollers 41a,41b,41c with the tube 132, and thus the points at which the tube 132 is compressed, to move along the tube 132, inducing a peristaltic pumping action on the elastomeric tube section 132.

In use, the blood collection vessel 11 is connected to a source of reduced pressure (not shown) and a suction tube 16 is used to collect blood from a surgical site, drawing it under reduced pressure into the blood collection chamber 11. To transfer blood from the blood collection chamber 11 to the blood concentration bag 14, one end of the blood transfer tube 13 is connected to an outlet port of the blood collection chamber 11 and the other end is connected to the inlet of the blood concentration bag 14. The peristaltic pump element 12 is fitted over the drive shaft (not visible in the drawings) that protrudes from the side wall of the shaker unit 15. Rotation of the drive shaft causes the peristaltic pump element 12 to draw blood from the collection chamber 11, through the blood transfer tube 13, and into the blood concentration bag 14.

The blood concentration bag 14 contains an inner bag made of porous polycarbonate, which contains a superabsorbent material. Plasma is drawn from the blood into the inner bag, leaving behind a blood cell concentrate that, after completion of the blood concentration process, can be transferred to a suitable receptacle for storage or for immediate retransfusion into the patient. The process of concentration of the blood typically takes between 5 and 30 minutes, after which the blood concentration bag 14 is disconnected from the blood transfer tube 13. Throughout the process, the blood concentration bag 14 is gently agitated by the shaker unit 15 to prevent occlusion of the pores in the inner bag. To achieve this, the blood concentration bag 14 is mounted on an oscillating table that forms part of the shaker unit 15.

If necessary, a second blood concentration bag 14 may be connected to the blood transfer tube 13 and the process repeated. At the end of the procedure, the blood transfer conduit, comprising the blood transfer tube 13 and the associated peristaltic pump element 12, is disconnected from the blood concentration bag 14, the blood collection chamber 11 and the shaker unit 15, and discarded.

1. A disposable blood transfer conduit for use in association with a pump drive unit, a blood collection vessel and a blood concentration device,
wherein the conduit comprises a blood transfer tube and a peristaltic pump element having an enclosed housing mounted about the blood transfer tube,
a first end of the blood transfer conduit being adapted for connection, in use, to the blood collection vessel and a second end of the blood transfer conduit being adapted for connection, in use, to the blood concentration device,
wherein at least part of the blood transfer tube is resiliently deformable and extends through the enclosed housing of the peristaltic pump element, and wherein the housing of the peristaltic pump element is adapted for releasable engagement, in use, with the pump drive unit to convey blood from the blood collection vessel to the blood concentration device.

2. The disposable blood transfer conduit of claim 1, wherein the blood transfer tube is formed from plastics material.

3. The disposable blood transfer conduit of claim 1, wherein the peristaltic pump element is such that, in use, the blood transfer tube is only partially occluded and not fully occluded.

4. The disposable blood transfer conduit of claim 1, wherein the housing of the peristaltic pump element defines a circular cavity with two openings that permit the housing to be fitted about the blood transfer tube.

5. The disposable blood transfer conduit of claim 4, wherein the openings are disposed such that the tube extends around the internal wall of the cavity, through at least 180° of arc.

6. The disposable blood transfer conduit of claim 4, wherein the housing has the form of a cylinder, with the two openings formed in the cylindrical surface.

7. The disposable blood transfer conduit of claim 4, further comprising compression elements arranged within the cavity such that, in use, they bear upon the resiliently deformable tube in order to exert a pumping action upon fluid contained within the tube.

8. The disposable blood transfer conduit of claim 7, wherein the compression elements are freely mounted rollers.

9. The disposable blood transfer conduit of claim 4, wherein the base of the housing comprises an aperture through which, in use, a drive shaft of the pump drive unit is inserted.

10. The disposable blood transfer conduit of claim 1, wherein the diameter of the blood transfer tube is between about 0.3 cm and 1.5 cm.

11. The disposable blood transfer conduit of claim 1, which is used in conjunction with a passive blood concentration device.

12. The disposable blood transfer conduit of claim 11, wherein the passive blood concentration device comprises a blood bag which contains a porous membrane and a superabsorbent material.

13. The disposable blood transfer conduit of claim 12, wherein the porous membrane forms an inner bag disposed within the blood bag and containing the superabsorbent material.

14. The disposable blood transfer conduit of claim 12, wherein the blood bag is formed of polyvinylchloride (PVC).

15. The disposable blood transfer conduit of claim 12, wherein the blood bag comprises a port by which it can be connected to the blood transfer conduit.

16. The disposable blood transfer conduit of claim 12, wherein the porous membrane is formed of porous polycarbonate.

17. The disposable blood transfer conduit of claim 12, wherein the pore size of the porous membrane is from about 0.01 μm to 1 μm.

18. A system for the concentration of blood for use in autologous blood transfusion, the system comprising a blood collection vessel and a blood concentration device, the blood collection vessel and blood concentration device being connected together by a disposable blood transfer conduit of claim 1.

19. The system of claim 18, which further comprises a pump drive unit for actuating the peristaltic pump element that forms part of the blood transfer conduit.

20. A method of concentrating blood for use in autologous blood transfusion, comprising collecting blood in a blood collection vessel, and transferring the blood from the blood collection vessel to a blood concentration device via a disposable blood transfer conduit according to claim 1.

21. A method of transferring blood between a blood collection vessel and a blood concentration device, comprising transferring the blood through a disposable blood transfer conduit according to claim 1.
